(19) United States
(12) Patent Application Publication
(10) Pub. No.: US 2022/0054703 A1
(43) Pub. Date: Feb. 24, 2022

(54) Implantable Materials and Uses Thereof

(71) Applicant: ASKEL HEALTHCARE OY, Helsinki (FI)

(72) Inventors: Virpi Muhonen, Helsinki (FI); Ilkka Kiviranta, Helsinki (FI); Elina Järvinen, Helsinki (FI); Minna Kellomäki, Kangasala (FI); Anne-Marie Haaparanta, Kangasala (FI); Ville Ellä, Pirkkala (FI)

(73) Assignee: ASKEL HEALTHCARE OY, Helsinki (FI)

(21) Appl. No.: 17/518,157

(22) Filed: Nov. 3, 2021

Related U.S. Application Data
(63) Continuation of application No. PCT/FI2015/050621, filed on Sep. 16, 2015.

(30) Foreign Application Priority Data
Sep. 17, 2014 (FI) 20145814

(51) Int. Cl. A61L 27/18; A61L 27/56; A61L 27/58; A61L 27/24; A61L 27/36

(52) U.S. Cl. CPC A61L 27/18; A61L 27/56; A61L 27/3654; A61L 27/24; A61L 27/58

(57) ABSTRACT

The present invention relates to a field of implants and more specifically to a three dimensional material comprising synthetic needle punched carded mesh and recombinant human collagen. Also, the present invention relates to uses of the three dimensional material. Furthermore, the present invention relates to medical devices comprising the implantable material of the invention.

### FIELD

The present invention relates to a field of implants and more specifically to a three dimensional material comprising synthetic needle punched carded mesh and recombinant human collagen. Also, the present invention relates to uses of the three dimensional material. Furthermore, the present invention relates to medical devices comprising the implantable material of the invention.

### BACKGROUND

Without blood supply and lymphatic drainage, articular cartilage stands isolated and virtually lacks the wound healing response of other connective tissues. Tissue's high exposure to biomechanical aberrations results in high incidence level of cartilage lesions. Such lesions, traumatic or due to prolonged non-physiological loading, often develop to osteoarthritis (OA) (Gelber A C, Hochberg M C, Mead L A, et al. Joint injury in young adults and risk for subsequent knee and hip osteoarthritis. Ann Intern Med. 2000; 133:321-8.). OA is the number one cause of musculoskeletal ailment worldwide, with the incidence level of 7-10% of people in western population. The estimated cost of OA in a newly diagnosed patient is $6,800 per year, thus postponing OA by 10 years leads to savings of $68,000 per patient (Le T K, Montejano L B, Cao Z, et al. Healthcare costs associated with osteoarthritis in US patients. Pain Pract. 2012; 12:633-40.). The expenditure for OA in EU is approximately €15-20 billion per year. While traditionally not indicated for the treatment of OA, cartilage repair has become a focus of increased interest due to its potential to alter the progression of the degenerative disease, with the hope of delaying or obviating the need for joint replacement.

In addition to significant morbidity and the potential for disablement, cartilage trauma and degeneration has major economic impacts as well. Estimating that the annual incidence of cartilage lesion is 23 per 100 000 population, there are more than 100 000 patients with a cartilage defect of the knee requiring repair treatment in the EU. The prevalence of cartilage pathologies is expected to rapidly increase in the following decades due to aging population as well as increased rate of obesity; the demand for knee replacements is projected to increase significantly through 2030. Young patients with symptomatic cartilage lesions represent a challenging population due to a combination of high functional demands and limited treatment options. The aim of articular cartilage repair treatment is to restore and maintain the normal function of the joint with repair tissue architecture indistinguishable of the natural hyaline cartilage. However, current repair techniques for cartilage lesions are inadequate and need development.

After surgical repair the biomechanical properties of the repaired site are weakened and postoperative loading has to be reduced. The lack of mechanical stimulus leads to slow tissue turnover and healing, thus, the recovery time remains long. Biomaterial scaffolds can provide structural support to the healing lesion to allow early load bearing and, thus, enhance the healing process. A wide variety of three dimensional scaffolds, both natural and synthetic, have been introduced for cartilage repair (Funayama A, Niki Y, Matsumoto H, et al. Repair of full-thickness articular cartilage defects using injectable type II collagen gel embedded with cultured chondrocytes in a rabbit model. J Orthop Sci. 2008; 13:225-32; Nooeaid P, Salih V, Beier J P, et al. Osteochondral tissue engineering: scaffolds, stem cells and applications. J Cell Mol Med. 2012; 16:2247-70, Sharma B, Fermanian S, Gibson M, et al. Human cartilage repair with a photoreactive adhesive-hydrogel composite. Sci Transl Med. 2013; 5:167ra6, Spiller K L, Maher S A, Lowman A M. Hydrogels for the repair of articular cartilage defects. Tissue Eng Part B Rev. 2011; 17:281-99, Wakitani S, Kimura T, Hirooka A, et al. Repair of rabbit articular surfaces with allograft chondrocytes embedded in collagen gel. J Bone Joint Surg Br. 1989; 71:74-80). Scaffolds can be divided into different physical forms, e.g., hydrogels, sponges and fibrous structures. Hydrogels resemble the native cartilage extracellular matrix with high water retention, but have low load-bearing capacity and mechanical strength. Fibrous structures can provide load-bearing capacity and mechanical strength, but are often associated with a relatively low cell-seeding efficiency, inadequate cell distribution, and an increase in chondrocyte dedifferentiation.

Currently there are three main methods applied to treat the knee articular cartilage damage: 1) marrow stimulation, such as microfracture technique, for small-sized lesions (≤2.5 cm²), 2) scaffold-assisted microfracture for middle-sized damages (2.5-4 cm²), and 3) cell-based therapy for large defects (≥4 cm²). The current clinical standard for cartilage repair of the knee is the microfracture technique, in which the subcartilage bone layer is punctured in order to release the reparative stem cells of bone marrow into the lesion site. This procedure is valid for lesions up to 2.5 cm², after which a scaffold is needed to support the fragile blood clot and augment the healing process. However, current scaffolds for cartilage repair are mostly thin membranes that function only as a cover to keep the blood clot at the repair site. Due to their limited biomechanical properties, post-operative loading has to be reduced, and the recovery time is long. Cartilage cell therapy has been used for focal cartilage lesion repair for 20 years and the method (autologous chondrocyte implantation, ACI, also called autologous chondrocyte transplantation, ACT) has demonstrated relatively good long-term outcomes (Vanlauwe J, Saris D B, Victor J, et al. Five-year outcome of characterized chondrocyte implantation versus microfracture for symptomatic cartilage defects of the knee: early treatment matters. Am J Sports Med. 2011; 39:2566-74, Harris J D, Siston R A, Pan X, et al. Autologous chondrocyte implantation: a systematic review. J Bone Joint Surg Am. 2010; 92:2220-33, Peterson L, Vasiliadis H S, Brittberg M, et al. Autologous chondrocyte implantation: a long-term follow-up. Am J Sports Med. 2010; 38:1117-24). However, the ACI technique is demanding, there still are uncertainties related to the clinical outcome, and the current high costs of these cell therapies do not meet the marginal benefits of the treatment. Hence, an unfortunate treatment gap exists for patients suffering from cartilage trauma.

There is a clear need for cost-effective, safe and reliable scaffolds that have a highly porous structure and an interconnected pore network supporting chondrocyte proliferation and cartilage matrix production that can be used both with microfracture and cell therapy techniques.

WO2007024125A1 describes a fibrous 3-dimensional scaffold, which is prepared via electro spinning method, and use of this scaffold for tissue regeneration. The scaffold is preferably prepared from polylactide acid (PLA).

Yamaoka H et al. (Journal of Biomedical Materials Research Part A 2009, pages 123-132, DOI: 10.1002/jbm.a.32509) used a combination of atelocollagen gel (including chondrocytes) and porous poly(L-lactide acid) (PLLA) scaffolds for cartilage tissue engineering and concludes that a hybrid scaffold has effective detainment of administered chondrocyte cells, good biocompatibility for the chondrocytes, and sufficient mechanical strength.

He X et al. (Tissue Engineering, 2010, Part C, Volume 16, Number 3, pages 329-338) developed a novel hybrid of PLLA and collagen sponge, wherein collagen sponge was enclosed in a cup-shaped PLLA sponge. The PLLA sponge cup was immersed in a collagen solution (porcine, type I) and vacuumed to fill the pores of the PLLA sponge with collagen solution. The central collagen sponge contributes to high porosity, and facilitates cell adhesion and distribution in the hybrid sponge.

Pulkkinen et al. (Osteoarthritis Cartilage, 2013, Volume 21, Number 3, pages 481-490) tested the repair of osteochondral defects with recombinant human type II collagen gel and autologous chondrocytes in rabbit. When the rhCol2 hydrogel was used to repair cartilage defects, the repair quality was histologically incomplete, but still the rhCol2 hydrogel repairs showed moderate mechanical characteristics and a slight improvement over those in spontaneous repair.

WO2013093921A1 describes an isolated fiber comprising of an internal synthetic polymer core (which can be biodegradable polymer, for example, PLA), coated with cellulose nanocrystals as intermediate layer and collagen as outer layer. These fibers can be further processed into different kinds of textiles with 3D structure and they can be used for tissue engineering scaffolds.

Haaparanta A-M et al. (J Mater Sci: Mater Med DOI 10.1007/s10856-013-5129-5) disclosed a study of collagen/polylactide acid (PLA) hybrid scaffold for cartilage tissue engineering. In this study synthetic 3D PLA carded mesh was combined with type I bovine dermal collagen in sandwich-like structure where the PLA carded mesh was on top and at the bottom of the scaffold.

The major limitation in the development of regenerative cartilage repair methods is the lack of appropriate biomaterial scaffolds fulfilling the physiological and mechanical properties required for suitable cartilage tissue engineering scaffold especially for repairing substantially large (4 cm²) lesions. The use of plain fiber scaffolds, for example different textiles, made of synthetic or natural biodegradable polymers are promising structures for cartilage tissue regeneration. However, these textiles are usually sparse structures with high porosity and large pores and are therefore not optimal for cell infiltration, attachment and even distribution. Even though the use of natural polymer alone mimics highly the natural environment in cartilaginous tissues, however, they lack the mechanical integrity needed for cartilage tissue engineering. Accordingly, there is still a need for scaffold structures that match dimensionally, mechanically and functionally to native cartilage.

### SUMMARY

The following presents a simplified summary in order to provide a basic understanding of some aspects of various embodiments of the invention. The summary is not an extensive overview of the invention. It is neither intended to identify key or critical elements of the invention nor to delineate the scope of the invention. The following summary merely presents some concepts of the invention in a simplified form as a prelude to a more detailed description of exemplifying embodiments of the invention.

In the present invention it was observed that by preparing a 3D hybrid scaffold including recombinant human collagen and needle punched carded mesh including one or more synthetic polymers, at least some disadvantages of the prior art scaffolds can be alleviated.

According to one aspect the present invention concerns a three dimensional material, preferably three dimensional porous material comprising a needle punched carded mesh comprising one or more synthetic polymers, and recombinant human collagen, preferably freeze dried recombinant human collagen.

According to another aspect the present invention concerns a medical device comprising a three dimensional material, preferably three dimensional porous material, comprising a needle punched carded mesh comprising one or more synthetic polymers, and recombinant human collagen preferably freeze dried recombinant human collagen.

According to another aspect the present invention concerns a medical device comprising a three dimensional material comprising needle punched carded mesh comprising one or more synthetic polymers, and recombinant human collagen for use in repairing a cartilage lesion or in postponing or eliminating the expansion of a cartilage lesion.

According to another aspect the present invention concerns a method of manufacturing a three dimensional material or a medical device comprising the same, the method comprising:

(i) obtaining carded mesh comprising one or more synthetic polymers;

(ii) needle punching the carded mesh to form a needle punched carded mesh;

(iii) obtaining recombinant human collagen; and

(iv) admixing the needle punched carded mesh and the recombinant human collagen and optionally freeze-drying.

According to another aspect the present invention concerns use of a three dimensional material comprising needle punched carded mesh comprising one or more synthetic polymers, and recombinant human collagen for producing a medical device. According to another aspect the present invention concerns a three dimensional material or a medical device comprising the same obtainable by a method comprising:

(i) obtaining a carded mesh comprising one or more synthetic polymers,

(ii) needle punching the carded mesh to form a needle punched carded mesh;

(iii) obtaining recombinant human collagen; and

(iv) admixing the needle punched carded mesh and the recombinant human collagen and optionally freeze-drying.

According to a further aspect the present invention concerns a method of repairing cartilage lesions, or postponing or eliminating the expansion of a cartilage lesion in a subject in need thereof, said method comprising

(i) providing a medical device comprising a three dimensional porous material comprising needle punched carded mesh comprising one or more synthetic polymers, and recombinant human collagen, and

(ii) applying the medical device to the lesion site of a subject.

Still, the present invention concerns a three dimensional material comprising needle punched carded mesh comprising one or more synthetic polymers, and recombinant human collagen for use in repairing cartilage lesions, or postponing or eliminating the expansion of a cartilage lesion in a subject.

A number of exemplifying and non-limiting embodiments of the invention are described in accompanied dependent claims.

Various exemplifying and non-limiting embodiments of the invention both as to constructions and to methods of operation, together with additional objects and advantages thereof, will be best understood from the following description of specific exemplifying embodiments when read in connection with the accompanying drawings.

The verbs “to comprise” and “to include” are used in this document as open limitations that neither exclude nor require the existence of also unrecited features. The features recited in depending claims are mutually freely combinable unless otherwise explicitly stated. Furthermore, it is to be understood that the use of “a” or “an”, i.e. a singular form, throughout this document does not exclude a plurality.

### BRIEF DESCRIPTION OF DRAWINGS

FIG. 1 shows an exemplary scanning electron microscopy image of the scaffold of the present invention wherein PLA fibres (white arrow) are embedded in freeze-dried recombinant human type II collagen (black arrow),

FIG. 2 shows exemplary relative expression of chondrogenic and cartilage markers of bovine chondrocytes cultured in a hybrid recombinant human collagen-PLA scaffold. Relative expression of type I collagen (Col1a1), type II collagen (Col2a1) and Sox9 transcription factor (Sox9) were analysed at days 2, 7 and 14 of chondrogenic culture and Day 0 cells were used as a control,

FIG. 3 shows an exemplary macroscopic image of repaired porcine knees with: A) the scaffold of the present invention, B) Chondro-Gide® membrane, and C) spontaneous healing without reparative construct after 4 months follow-up,

FIG. 4 shows an exemplary microscopic structure of the repair tissue of the cell-laden scaffold of the present innovation in an orthotopic porcine model after 4 months follow-up,

FIG. 5 shows an exemplary histological detection of proteoglycans of the repair tissue by SafraninO staining representing average staining results of A) the present innovation, B) Chondro-Gide® membrane and C) spontaneous healing,

FIG. 6 shows confocal microscopy images of needle punched PLA96/4 mesh (PLA) and needle punched PLA96/4 mesh together with freeze-dried collagen (COPLA). The white dots represent cell nuclei. Samples were imaged from both sides (side A and side B). Cell suspension with 500 000 cells/sample disk of 8 mm in diameter was pipetted on side A. PLA n=4, COPLA n=3,

FIG. 7 shows a scanning electron microscopy image of chondrocytes adherence to the collagen component of the scaffold of the present invention, and

FIG. 8 shows a plastic embedded sample of the needle punched PLA96/4 mesh together with freeze-dried collagen. The black lines are wrinkles of the thin plastic section, grey dots represents the chondrocyte nuclei. Cell suspension with 500 000 cells/sample disk of 8 mm in diameter was pipetted on one side (upper surface), nevertheless, the cells can be found throughout the material structure.

### DESCRIPTION

The present invention concerns a three dimensional material including needle punched carded mesh made of, or including, one or more synthetic polymers, and recombinant human collagen. As defined herein, carded mesh is a mesh obtainable by carding that is a mechanical process that disentangles, cleans and intermixes fibres to produce a continuous randomly oriented web, i.e., a carded mesh. Carding breaks up locks and unorganised clumps of stapled fibres and then aligns the individual fibres to be mostly separated from each other.

As defined herein the needle punched carded mesh is a mesh obtainable by needle punching a carded mesh. Needle punching is a process that uses needles with notches along the shaft of the needle that grabs the top layer of fibers and tangles them with the inner layers of fibers as the needle enters the fibers. Since these notches face down towards the tip of the needle, they do not pull the fibers out as the needle exits the card. Needle punching creates tangled and compressed felt from card and improves the mechanical properties still leaving the structure highly porous.

As used herein, “a three dimensional material” refers to any material that has height, width and depth. One example of three dimensional structures is a scaffold.

The three dimensional material of the present invention is preferably implantable, biodegradable and biocompatible.

As defined herein, biodegradable material is a material, which after introduction into the body requires no retrieval or further manipulation because it is degraded into soluble and non-toxic by-products.

As defined herein, implantable material is a material of any shape or size, which is suitable for implanting to a subject.

As defined herein, biocompatible material is a material that is not harmful or toxic to living tissue.

According to one embodiment, the needle punched carded mesh is processed by using biodegradable and biocompatible polymer fibers comprising or made of one, two or several synthetic polymers. Two or several synthetic polymers may be utilized for example in two ways: 1) by producing the fibers using polymer blends and/or copolymers or, 2) by mixing fibers made of different polymers. Examples of suitable synthetic polymers include but are not limited to polyesters, polyglycolic and polylactic acid (PLAs) homopolymers and copolymers, glycolide and lactide copolymers and polycaprolactones. In one embodiment the synthetic polymer or polymers is/are selected from the group consisting of polyglycolide (PGA), poly(lactide-co-glycolide) (PLGA), polylactide (PLA), poly(caprolactone) (PCL) and poly(lactide-caprolactone) (PCLC), diol/diacid aliphatic polyester, polyester-amine/polyester-urethane, poly(valerolactone), poly hydroxy alkanoates, poly(hydroxyl butyrate) and poly(hydroxyl valerate). Preferable synthetic polymers are polylactides. According to one embodiment, the needle punched carded mesh comprises only one synthetic polymer or is made of only one synthetic polymer.

As used herein “fibrous” refers to a material made of fibers. Fibers having diameters of only one size or different sizes may be used in the needle punched carded mesh of the present invention. These polymer fibers may be selected from polymer fibers having a diameter of 5-100 μm, more specifically 10-30 μm. In one embodiment, the needle punched carded mesh comprises fibres having diameter of from 5 to 100 μm. The diameters are average diameters of the fibers in the structure. The cross-section of the fiber is not limited only to a round one, but may also be any other shape such as oval, star-shaped, right-angle or triangle.

In one embodiment of the invention porosity of the needle punched carded mesh of the present invention is at least 85%. Exemplary porosities are 85%, 86%, 87%, 88%, 89%, 90%, 91%, 92%, 93%, 94%, 95%, 96%, 97%, 98% and 99%. Accordingly, in one embodiment of the invention the hybrid of the needle punched carded mesh and/or the collagen material have the porosity of 85-99%. As defined herein, the porosity, i.e., a void fraction is a measure of the void (i.e., “empty”) spaces in a material, and is a fraction of the volume of voids over the total volume. In a specific embodiment, the three dimensional material of the invention is a porous structure with pore network throughout the material.

In one embodiment of the invention the thickness of the three dimensional material is from 0.1 to 50 mm.

According to one embodiment the needle punched carded mesh is combined with recombinant human collagen. Suitable human collagens may vary in their amino acid sequence, their chain length and/or folding as long as they retain their capability to induce or support the formation of functional cartilage extracellular matrix. According to the present invention the combination of synthetic polymer fibrous mesh with collagen can enhance the biological signalling of the cells compared to, e.g., fibrous structure of synthetic polymers. In a specific embodiment, the use of collagen, preferably freeze-dried collagen, inside the fibrous mesh enhances the entanglement of the cells and also promotes the new tissue formation.

As defined herein, recombinant human collagen refers to a human collagen polypeptide, which is produced by using recombinant techniques, e.g. using appropriate polynucleotides, expression vectors and host cells. Recombinant techniques are well known to a person skilled in the art and for example several commercial recombinant human collagens are present on the market.

Use of recombinant human collagen lowers the risks of transmitting known and unknown animal-derived pathogens and undesirable immunological responses. In addition, unlike other naturally-derived materials for cartilage regeneration, the recombinant human collagen does not suffer from batch-to-batch variability. Accordingly, recombinant human collagens can be produced in a grade required by good manufacturing practices (GMP), in high amounts and of uniform quality.

A recombinant human collagen may be selected from the group consisting of recombinant human collagen types I, II, III, V, VI, IX and XI. Any combination of these collagen types may also be utilized. In one embodiment the recombinant human collagen is recombinant human collagen type I, II or III, more specifically recombinant human collagen type II or III. In another specific embodiment, recombinant human collagen is a combination of at least recombinant human type I, II and III collagens, at least recombinant human type I and III collagens, at least recombinant human type I and II collagens, or at least recombinant human type II and III collagens.

As used herein “the recombinant human collagen material” refers to any material (e.g. any gel) comprising recombinant human collagen. According to one embodiment, the recombinant human collagen material is porous (i.e., comprises pores). For example freeze-drying makes the collagen porous and elastic and thus well suitable for its purpose, e.g., to support chondrocyte proliferation and cartilage matrix production. Collagen such as freeze-dried collagen network, is an excellent microenvironment for cell attachment. In one embodiment of the invention the recombinant human collagen is freeze-dried. The collagen (e.g. in the form of collagen solution) may be freeze-dried as such. Pore size of the collagen structure varies between 20-250 μm, and can be selected from 20-250 μm, 50-250 μm, 30-200 μm, 40-200 μm, 50-200 μm, or 60-200 μm. Also, it is possible to convert the collagen into a gel before freeze-drying, i.e. the collagen(s) may be in the form of a freeze-dried gel.

After freeze-drying, the collagen material may still be cross-linked. In one embodiment of the invention the recombinant human collagen is cross-linked. Suitable cross-linking methods are well known to a person skilled in the art and include but are not limited to the use of chemical cross linking agents such as to 1-ethyl-3-(3-dimethylaminopropyl)-carbodiimide, glutaraldehyde, genipin, and also UV light.

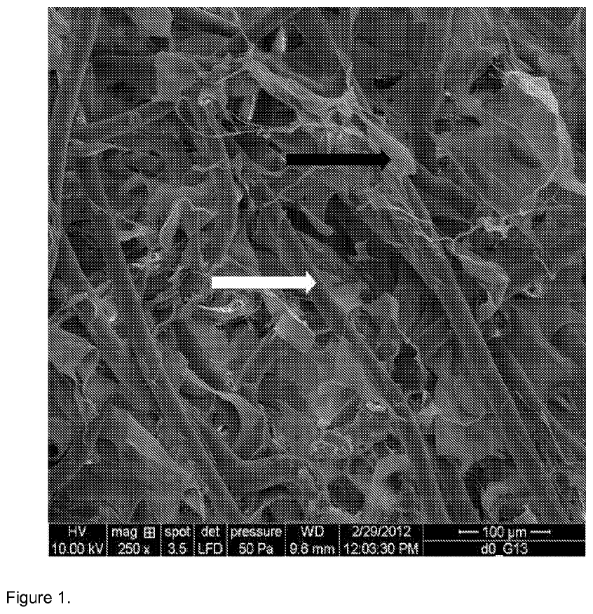

An exemplary scanning electron microscopy image of a scaffold (i.e., three dimensional material) according to the present invention including PLA fibres embedded with freeze-dried recombinant human type II collagen is shown in FIG. 1.

The three dimensional material of the present invention may also include materials or agents not described in this disclosure but which are well known to a skilled artisan. These material or agents may be selected for example from agents capable of promoting chondrogenesis, differentiation of chondrocytes, inhibition of dedifferentiation of chondrocytes, synthesis and the three-dimensional arrangement of extracellular matrix components, and/or stable hyaline cartilage formation [for example growth factors (e.g., TGF-beta)].

According to one embodiment the present invention concerns a medical device comprising a three dimensional material comprising a needle punched carded mesh made of, or comprising one or more synthetic polymers, and recombinant human collagen. As defined herein, a medical device is an instrument, apparatus, implant, in vitro reagent, or similar or related article that is used to diagnose, prevent, or treat disease or other conditions. The medical device of the present invention is implantable and biocompatible and in a specific embodiment also biodegradable.

According to one embodiment the medical device of the present invention comprises cells. According to a particular embodiment, the medical device comprises cells capable for chondrogenesis and/or cartilage formation, e.g., cartilage cells and/or mesenchymal stem cells. In a specific embodiment, the mesenchymal stem cells are enriched from bone marrow and/or differentiated bone marrow mesenchymal stem cells. As defined herein “differentiated bone marrow mesenchymal stem cells” refers to chondroprogenitor cells or chondrocytes. The cells, such as cartilage cells, mesenchymal stem cells or a combination thereof can be applied to the medical device either prior to or after implantation.

As defined herein “mesenchymal stem cells” refers to multipotent stromal cells that can differentiate into a variety of cell types (e.g., chondrocytes). Mesenchymal stem cells may be isolated for example from bone marrow, synovium, fat tissue and/or cartilage by any known isolation method known in the art. Mesenchymal stem cells are multipotent cells present in mesenchymal tissues. Mesenchymal stem cells may be either autologous (mesenchymal stem cells from the individual to be treated) or allogenic (mesenchymal stem cells from another individual belonging to the same species). Mesenchymal stem cells used in the scaffold of the present invention may be from one or several tissues.

The highly anisotropic structure of matrix components of mature articular cartilage responsible for its mechanical competence is synthesized and degraded by cells called chondrocytes, which can have different phenotypes depending on the growth stage or age of the individual. Therefore, as used herein “cartilage cells” refers to chondrocytes. Chondrocytes may be either autologous (chondrocytes from the subject to be treated) or allogenic (chondrocytes from another subject belonging to the same species).

Cartilage is classified in three types, elastic cartilage, hyaline cartilage and fibrocartilage. Cartilage is composed of specialized cells called chondrocytes that produce a large amount of extracellular matrix composed of collagen fibres and abundant ground substance rich in proteoglycans.

As used herein “cartilage lesions” refers to any defects of the cartilage. These lesions can be caused for example by congenital cartilage defects (e.g., chondrodystrophies), trauma, age or age-related diseases, inflammatory diseases (e.g., costochondritis), osteoarthritis, rheumatoid arthritis, psoriatic arthritis, or related autoimmune diseases (e.g., relapsing polychondritis), septic arthritis or other diseases (e.g., achondroplasia, spinal disc herniation, tumors (either benign or malignant) made up of cartilage tissue). Patients having cartilage lesions may become severely limited due to pain, reduction of joint motion, and deterioration of morphological integrity. In one embodiment of the invention the cartilage lesions are articular cartilage lesions.

The medical device of the present invention is favourable for cells, i.e., the implantable material supports the repair of the lesion. Accordingly, in one embodiment of the invention the device possesses high porosity, preferably at least 85%, and interconnected pores of suitable sizes for optimal environment for cell migration and viability, and nutrition exchange.

According to one embodiment the present invention concerns a medical device including three dimensional material comprising a needle punched carded mesh made of or comprising one or more synthetic polymers, and recombinant human collagen and in a specific embodiment also cells, for use in repairing cartilage lesions or in postponing or eliminating the expansion of a cartilage lesion.

Use of the cell-based medical device involves several procedures, that is, e.g., the harvesting of a bone marrow or cartilage biopsy, the isolation and proliferation and optionally also differentiation of stem cells or chondrocytes in vitro, and the subsequent transplantation of cells into the lesions either before, simultaneously (i.e., in the device), or after transplantation of the medical device.

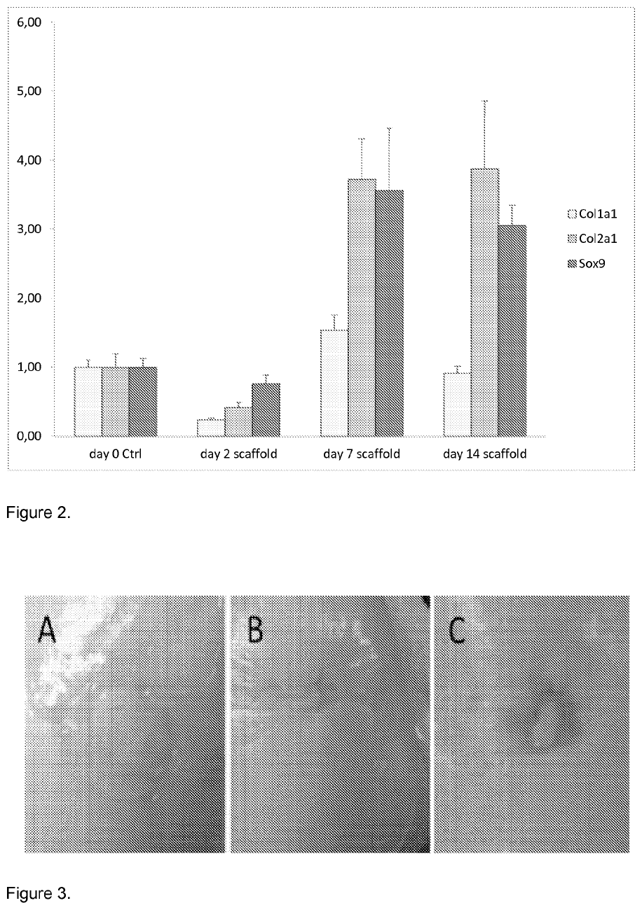

Cell culture experiments and messenger RNA expression analysis of the experimental section show that when chondrocytes are cultured in the scaffold of the present invention, the cells start to upregulate chondrogenic and cartilage markers (FIG. 2). In an large animal model (i.e., a porcine knee lesion model) the scaffold of the present invention together with autologous cartilage biopsy-based chondrocytes was shown to produce articular cartilage tissue of good tissue structure and biomechanical properties (FIG. 3-5).

Chondrocyte cells can be seeded to the scaffold for example by applying (e.g., pipetting or applying by bioreactors) the cell suspension on the scaffold. In one embodiment of the invention the adequate cell density for cartilage production, highly relevant parameter for clinical applicability, may be important. The cell density of chondrocytes for cartilage production in the scaffold of the present invention is not limited to, but can be selected from the group consisting of 10³-10⁸, 10⁴-5×10⁷, 10⁵-10⁷ cell/cm² or 10²-10⁷, 10³-5×10⁶, 10⁴-10⁶ cell/cm³.

The medical device of the present invention is particularly suitable for repairing cartilage lesions, or postponing or eliminating the expansion of a cartilage lesion.

In order to be more effective and more suitable for large lesions (i.e., larger than 4 cm²) the device may be provided with further factors capable of repairing the tissue or enhancing growth of the repair tissue. In one embodiment of the invention the medical device further comprises an agent capable of promoting chondrogenesis, differentiation of chondrocytes, inhibition of dedifferentiation of chondrocytes and stable hyaline cartilage formation.

According to one embodiment the present invention concerns a method for producing a three dimensional material or a medical device comprising the same, the method comprising:

(i) obtaining carded mesh comprising one or more synthetic polymers,

(ii) needle punching the carded mesh to form a needle punched carded mesh;

(iii) obtaining recombinant human collagen; and

(iv) admixing the needle punched carded mesh and the recombinant human collagen and optionally freeze-drying.

The fibres for carded mesh may be produced by melt spinning of synthetic bioabsorbable polymer (examples of suitable synthetic materials include polyesters, polyglycolide (PGA), and polylactide (PLA) homopolymers and copolymers of lactides, glycolide and lactide copolymers and caprolactones). In one embodiment, the fibres of the implantable material are produced by using a melt-spinning technique, which is well known to a person skilled in the art. In melt spinning techniques solvents are not needed. The fibres may be non-oriented, online oriented or oriented in a separate process. The fibres may have a cross-sectional shape other than conventional round shape, the fibres may have longitudinally oriented sectors of different polymers and the fibres may take another form in liquid or inside the body. In one embodiment, the fibres may split due to the sectors of different polymers used to produce the fibres.

The carded mesh used to manufacture the needle punched carded mesh is produced of polymer fibres. In one embodiment, poly-(L,D) lactide with 96% of L-lactide and 4% of D-lactide fibres are used. The fibres may be produced of homopolymers or copolymers. They may also be produced from polymer blends. The fibres can be used as straight, plain fibres or the fibres may be textured, crimped or heat threated. The produced fibres are always cut into staple fibres and subsequently carded into fibrous mesh, and afterwards needle punched. The card and therefore also the needle punched carded mesh can be produced also using fibres made of different polymers. Then all the fibres are cut to the staple fibres, mixed together and used to produce carded mesh which is further needle punched.

The needle punching method as used herein, is a method of joining the carded mesh fibres together by a needle such as a barbed needle or in machine by a needle bed comprising one or several needles such as barbed needles to form an interlocking structure, and this method can also be any other method where the fibres are mechanically entangled together to form a structure with a desired porosity and mechanical properties comprising of staple fibres.

According to one embodiment of the invention the polymeric needle punched carded mesh fibres, any optional agents and recombinant human collagen, and optionally cells are combined. According to an exemplary embodiment a solution including recombinant human collagen is impregnated into needle punched carded mesh followed e.g., by freeze-drying. In one embodiment the needle punched carded mesh is filled in with recombinant human collagen to acquire the scaffold. According to one embodiment the freeze-drying is omitted. In addition to freeze drying or alternatively, in another embodiment the human recombinant collagen may be made porous by other processing methods than freeze-drying, for example with gas aided processing.

According to a particular embodiment the collagen component is cross-linked to increase the structure stability.

Before classifying a human or animal patient as suitable for the therapy of the present invention, the clinician may examine a patient. Based on the results deviating from the normal and revealing a cartilage lesion, the clinician may suggest the three dimensional material or medical device of the invention for a patient. The three dimensional material or the medical device may be applied to the lesion site of a subject, e.g., a mammal or human subject, for purposes which include not only complete cure but also prophylaxis, amelioration, or alleviation of disorders or symptoms related to a cartilage lesion. In one embodiment of the invention, the subject is a human patient or an animal. In another embodiment of the invention, the lesion site is selected from elastic cartilage, hyaline cartilage and fibrocartilage. In a specific embodiment of the invention the cartilage is in any synovial joint of the body, particularly selected from the group consisting of, e.g., a hip, knee, ankle, elbow, shoulder, finger, toe, wrist and temporomandibular joint.

Doctors or clinicians may use the soft biodegradable implant material of the present invention in open surgical procedures as well as minimally invasive surgical procedures. For example, the material can be endoscopically implanted at a lesion site (e.g., during arthroscopic surgery).

Therapeutic effect may be assessed for example by monitoring the symptoms of a patient and/or the size of a lesion in the patient. The terms “repairing”, “postponing” or “eliminating” as used herein, do not necessarily imply 100% or complete repair, postpone or elimination. Rather, there are varying degrees of which one of ordinary skill in the art recognizes as having a potential benefit or therapeutic effect. In this respect, the present invention can provide any amount or any degree of repair, postpone or elimination of a lesion.

It will be obvious to a person skilled in the art that, as the technology advances, the inventive concept can be implemented in various ways. The invention and its embodiments are not limited to the examples described above but may vary within the scope of the claims.

### EXPERIMENTAL

### Comparative Example 1

A carded mesh structure of the scaffold described in Haaparanta et al. (J Mater Sci: Mater Med DOI 10.1007/s10856-013-5129-5) was prepared as follows. The melt spun online oriented poly(L/D)lactide 96/4 (PLA96/4) (Purac Biochem, Gorinchem, The Netherlands) fibers with diameter of single fiber ˜20 μm were cut into staple fibers and carded with manual carding machine. The fibers lay randomly on top of each other in the process to form carded mesh. The carded mesh was subsequently gamma sterilized at 25 kGy. The (bovine dermal type I) collagen (PureCol®, Nutacon B.V., Leimuden, The Netherlands) solution was made into gel by adjusting the pH of the collagen solution to 7.20. The concentration of the used collagen gel was 0.5 wt %. The carded PLA96/4 mesh was laid on top of and at the bottom of collagen solution in the structure, i.e., so-called sandwich structure with three distinct layers, where the porous collagen structure contained carded PLA96/4 mesh layers on top and on the bottom of the scaffold. The scaffolds were frozen at −30° C. for 24 h and freeze-dried for 24 h. The scaffolds were further cross-linked with 95% ethanol solution with 14 mM EDC and 6 mM NHS (Sigma-Aldrich, Helsinki, Finland), washed and freeze-dried as described earlier. The methods are also described in Haaparanta et al.

The mechanical strength (Stiffness [N/mm]) of the scaffold was tested in wet conditions (scaffolds immersed in PBS for 24 h, 37° C.) with compression rate of 0.5 mm/min and cell load of 1 kN with Lloyd LR30K mechanical tester (Lloyd Instruments Ltd, Hampshire, UK). The stiffness of the scaffold (65.1±10.9 N/mm) was found to be too weak for cartilage tissue engineering, i.e., the structure was not retained when compression force was applied.

### Comparative Example 2

The carded mesh of Haaparanta et al. (J Mater Sci: Mater Med DOI 10.1007/s10856-013-5129-5) was prepared but by using a homogenous hybrid structure by loading the highly porous recombinant human collagen type II (Fibrogen Europe, Ltd., Helsinki, Finland) throughout with the carded mesh. Prior the recombinant human collagen solution was made into gel by adjusting the pH of the collagen solution to 7.20. The carded PLA96/4 mesh was immersed throughout with the collagen solution and the structure was subsequently freeze-dried (frozen at −30° C. for 24 h and freeze-dried for 24 h as described earlier and in Haaparanta et al.) to form homogenous hybrid structure. The scaffolds were further cross-linked with 95% ethanol solution with 14 mM EDC and 6 mM NHS (Sigma-Aldrich, Helsinki, Finland), washed and freeze-dried as described earlier (and in Haaparanta et al.).

The stiffness of the scaffold was tested as described in Comparative Example 1 and was found to be improved (107.12±24.72 N/mm) compared to the sandwich structure scaffolds in Comparative Example 1, i.e., the structure of the scaffold was better retained when compression force was applied.

### Comparative Example 3

The three dimensional material of Comparative Example 2 was tested in an orthotopic animal model. A cartilage lesion was created in a porcine knee and the cartilage tissue was harvested for cell isolation and proliferation procedure in vitro. In a second operation, the lesion was debrided and the hybrid scaffold loaded with autologous chondrocytes was sutured to the lesion site. Briefly, the animals were set on a supine position on the operating table and a medial parapatellar arthrotomy was made on the right hind leg. The patella was dislocated laterally, and the articular part of the femur was exposed. A single circular chondral lesion, 8 mm in diameter with the whole depth of the joint cartilage, was created in the medial condyle of the femur. The harvested cartilage was placed in sterile phosphate-buffered saline solution (PBS) and stored for further processing for no more than 12 hours at 4° C. The cartilage samples were minced and digested overnight in type 2 collagenase solution. The yielded chondrocytes were cultured until passage 2 and stored at −140° C. until the repair operation. The cartilage repair operation was performed three weeks after the biopsy operation. The cultured chondrocytes were thawed, calculated, suspended in culture media and transported into the operating room. The joint was approached through the previously used incision. The lesion was debrided from scar tissue. Subsequently, the scaffolds were sutured into the surrounding healthy cartilage and the 0.2 ml cell suspension was injected into and under the 8 mm-in-diameter scaffold. Fibrin glue was used to seal the constructed area. The animals in the control group were left without reparative constructs. After 10 weeks, the animals were sacrificed and tissue samples collected. The histological analysis revealed that the tissue engineered structure had submerged into the subchondral bone and the cartilage tissue was left unrepaired.

### Example 1

The carded PLA96/4 mesh of Comparative Example 2 was needle punched followed by admixing with highly porous recombinant human collagen type II (Fibrogen Europe, Ltd., Helsinki, Finland).

The needle punching method uses needles with specific barbs that are chosen on the basis of the used fibre. By moving the card or the bed of needles in perpendicular direction or at a chosen angle against or along the card, the barbs take on the fibres and pull and push the fibres through the network of fibres. This results in the entanglement of the fibres in the network and a needle punched carded mesh structure is formed with mechanical interlocking of the fibres thus forming a structure for the medical device. The carded needle punched PLA96/4 mesh was subsequently gamma sterilized at 25 kGy. The recombinant human collagen solution was made into gel by adjusting the pH of the collagen solution to 7.20. The carded needle punched PLA mesh was immersed throughout with the collagen solution and the structure was subsequently freeze-dried (frozen at −30° C. for 24 h and freeze-dried for 24 h) to form homogenous hybrid structure. The scaffolds were further cross-linked with 95% ethanol solution with 14 mM EDC and 6 mM NHS (Sigma-Aldrich, Helsinki, Finland), washed and freeze-dried as described earlier.

The stiffness of the scaffold was found to be further improved (122.17±1.61 N/mm) compared to the homogenous hybrid structure of scaffolds with carded mesh in Comparative Example 2, i.e., the structure was retained when compression force was applied.

TABLE 1
Comparison of the strength of the 3D structures

| 3D structure | Retain ability/stiffness of the scaffold |
|---|---|
| Comparative Example 1 | − |
| Comparative Example 2 | + |
| Example 1 | ++ |

### Example 2

Bovine chondrocytes were seeded into hybrid recombinant human collagen type II-PLA96/4 scaffolds of Example 1. The scaffolds containing the cells were cultured in chondrogenic cell culture conditions up to two weeks. RNA was isolated from the samples at day 0, 2, 7 and 14 for real-time quantitative PCR (qRT-PCR) analysis of type I and II collagens, and Sox9 transcription factor (TaqMan probes and program, COL1A1: Bt03225322_m1; COL2A1: Bt03251861_m1; SOX9: AIVI3LK, Applied Biosystems, USA). The results demonstrated that the basic connective tissue marker of type I collagen was expressed at similar levels both in control substrate and hybrid scaffold throughout the 14-day culture. Moreover, the chondrogenic and cartilage marker of Sox9 transcription factor and the cartilage specific structural protein of type II collagen were clearly up-regulated in the hybrid scaffold (FIG. 2). This study showed that the hybrid scaffold of PLA96/4 mesh interlocked with the freeze-dried recombinant human collagen has the potential to stimulate markers crucial for cartilage formation in vivo.

### Example 3

Test scaffold: Hybrid recombinant human collagen type II—PLA96/4 scaffold of Example 1.

Control scaffold: The Chondro-Tide® scaffold (Geistlich Pharma AG) is a two-layer hydrophilic collagen type I/III membrane extracted from pigs.

The right knee of a pig (Sus scrofa domestics) was opened through a lateral parapatellar arthrotomy and a single circular chondral lesion with a diameter of 8 mm was created in the medial condyle of femur. The cartilage was harvested from the created lesion. Chondrocytes were isolated, expanded and stored at −140° C. until the repair operation that took place three weeks after the first operation. Prior to the repair operation, the scaffolds were loaded with the cells. The biopsied knees were reoperated and the cartilage lesion was cleaned and repaired with the cell-laden test or control scaffold (bilayer porcine-derived collagen film, Chondro-Gide® membrane). Other animals served as spontaneous repair controls, where the cleaned lesion was left untreated. The animals were sacrificed after four (4) months and tissue samples collected for macroscopical, biomechanical, micro computed tomographical, histological and immunohistochemical analyses.

FIG. 3 shows macroscopic image of repaired knees with: A) the scaffold of the present invention, B) Chondro-Gide® membrane, and C) spontaneous healing without reparative construct after 4 months follow-up. The repair tissue of the cartilage lesions treated with the scaffold of the present invention was healthy cartilage-like white homogenous tissue well-aligned to the surrounding healthy cartilage surface (A), whereas the macroscopic tissue appearance of the type I/111 collagen bilayer matrix repaired lesions was uneven and typically presented “sunken” repair tissue submerged into the subchondral bone (B). The spontaneously healed lesions represented well repaired tissue on average, but some surface structure aberrations were present (C).

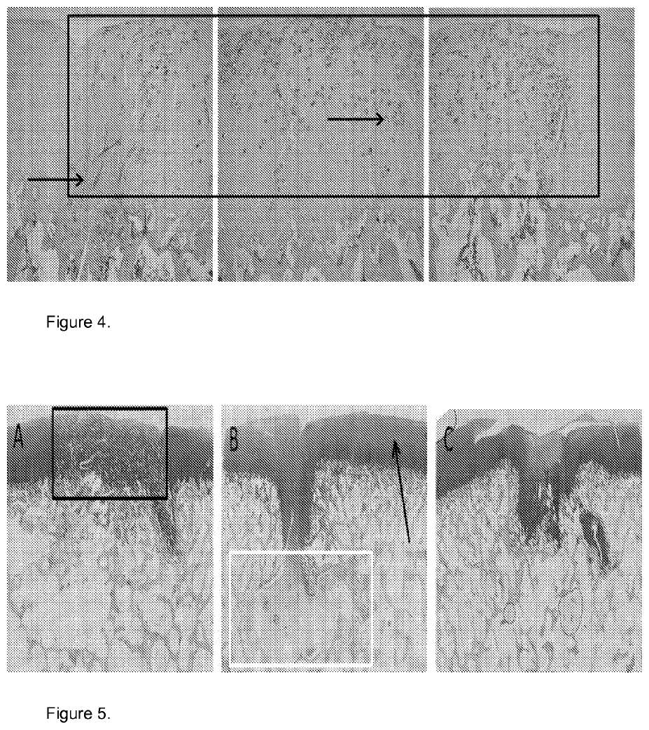

FIG. 4 shows microscopic structure of the repair tissue of the cell-laden scaffold of the present innovation in an orthotopic porcine model after 4 months follow-up. The PLA96/4 fibres (black arrows) are still visible in the repair tissue (black box). The repair tissue is well integrated and aligned with the host cartilage tissue.

FIG. 5 shows histological detection of proteoglycans of the repair tissue by SafraninO staining. The images represent the average staining results of A) the present invention B) Chondro-Gide® membrane and C) spontaneous healing. Black box highlights the repair site. The white box represents the typical subchondral bone reaction, where the cell-laden scaffold is submerged into the bone structure. The native cartilage tissue is indicated by arrow.

The results demonstrated that the present invention has good macroscopic tissue quantity and quality (FIG. 3), good repair tissue alignment parallel to native cartilage (FIG. 4) and good extracellular matrix deposition of proteoglycans (FIG. 5). The properties of the present invention, i.e., the mechanically cartilage-friendly needle punched PLA96/4 mesh together with a hydrophilic polymer network of recombinant collagen is able to imbibe high amount of water mimicking the glycosaminoglycan polymers entwined with collagen network of articular cartilage matrix. Thus, the carded needle punched PLA96/4 mesh and the collagen component together make the scaffold of the present invention considerably more suitable for cartilage reconstruction than the bilayer structure of the porcine-derived collagen film of the state-of-the-art scaffold. Moreover, the present invention is animal product free and does not suffer from batch-to-batch variation.

Thus, the difference in the PLA96/4 structure (i.e., carded mesh vs. needle punched carded mesh) of the homogenous hybrid scaffold was shown to have a surprisingly large effect on cartilage tissue healing. The results demonstrate that the scaffold of the present invention provided a cartilage-friendly surrounding that supported the tissue healing. Strikingly, only after 4 months of repair surgery, the histological results revealed completely healed cartilage surface with structural and mechanical properties comparable to native cartilage tissue. The most critical parameters of the repair tissue are listed and compared between the scaffold of the present invention and the type I/III collagen bilayer matrix in Table 2.

TABLE 2
Comparison of the most critical parameters of the repair tissue
after 4 months of healing. Chondral lesions of 8 mm in diameter
in porcine knee were treated with the scaffold of the present
invention or with type I/III collagen bilayer matrix.

| | Hybrid recombinant human | Type I/III |
|---|---|---|
| | collagen-PLA96/4 | collagen |
| | scaffold | bilayer matrix |
| Macroscopic repair | +++ | ++ |
| tissue fill | | |
| Proteoglycan content | +++ | ++ |
| of repair tissue | | |
| Type II collagen content | ++ | + |
| of repair tissue | | |
| Biomechanical properties | +++ | ++ |
| of repair tissue | | |
| Severity of subchondral | + | +++ |
| reaction¹ | | |

¹+ refers to mild, ++ moderate and +++ significant subchondral bone reaction.

### Example 4

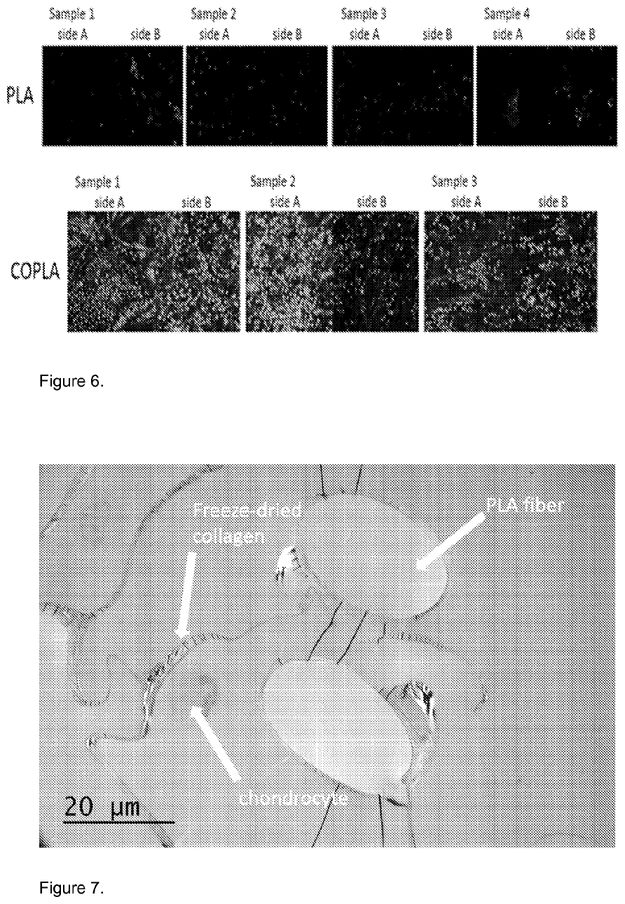

Bovine chondrocytes were seeded into scaffold disks of 8 mm in diameter comprising either of needle punched PLA96/4 mesh (PLA) or needle punched PLA96/4 mesh together with freeze-dried collagen (COPLA). The chondrocytes (500 000 cells in 40 μl on media) were seeded only on one side of the scaffold (“A”). The cells were let to attach for 2 minutes in room temperature, fixed with 10% formalin, washed with phosphate-buffered saline (PBS), and stained with nuclei stain (Hoechst 33342 0.5 μg/ml) for 5 minutes at room temperature. After staining, the samples were washed with PBS and stored in water at +4° C. until imaging with confocal microscope (Leica TCS CARS SP8). Confocal z-stacks (average depth of 290 μm) were collected and maximum projection images were created to demonstrate the total cell amount. FIG. 6 shows representative maximum projection images of total cell amount on both sides (“A” and “B”) of the scaffolds investigated (PLA n=4, COPLA n=3). The white dots represent cell nuclei. Cell adhesion is drastically better when the needle punched PLA96/4 mesh is combined with freeze-dried collagen.

### Example 5

Bovine chondrocytes (500 000 cells) were seeded into scaffold disks of 8 mm in diameter comprising of needle punched PLA96/4 mesh together with freeze-dried collagen. The cells were let to attach for 2 minutes in room temperature, fixed with 10% formalin, washed with phosphate-buffered saline (PBS) and dehydrated in absolute ethanol. Dehydrated specimens were dried using critical point dehydration, mounted into aluminum stubs and coated with platinum. Samples were cut into 60 nm thick sections and imaged in scanning electron microscope. Chondrocytes prefer adhesion to collagen component over the PLA96/4 fibers, as shown in FIG. 7.

### Example 6

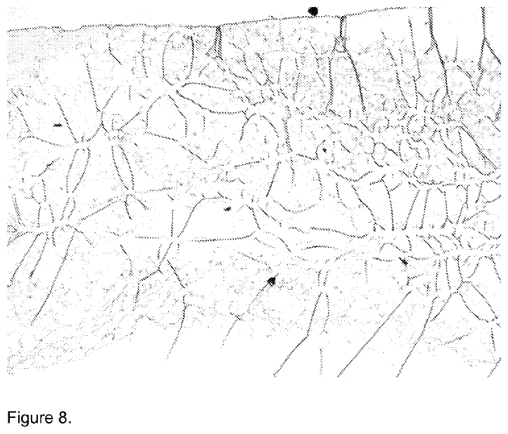

Bovine chondrocytes (500 000 cells) were seeded into scaffold disks of 8 mm in diameter comprising of needle punched PLA96/4 mesh together with freeze-dried collagen. The cells were seeded only on one side of the scaffold and let to attach for 2 minutes in room temperature, fixed with 10% formalin, washed with phosphate-buffered saline (PBS) and dehydrated in absolute ethanol. Samples were plastic embedded and 600 nm thick sections were cut and mounded on to glass coverslips. The sections were stained with standard hematoxylin and eosin staining for light microscopic imaging. Due to open porosity of the hybrid scaffold structure, the cells can be found throughout the scaffold as demonstrated in FIG. 8. The black lines are wrinkles of the thin plastic section, grey dots represents the chondrocyte nuclei.

1. A three dimensional porous material comprising needle punched carded mesh comprising one or more synthetic polymers, and recombinant human collagen.

2. The material according to claim 1 wherein the material is biodegradable.

3. The material according to claim 1 wherein the synthetic polymer or polymers is/are selected from the group consisting of polyglycolide (PGA), poly(lactide-co-glycolide) (PLGA), polylactide (PLA), poly(caprolactone) (PCL) and poly(lactide-caprolactone) (PCLC), diol/diacid aliphatic polyester, polyester-amine/polyester-urethane, poly(valerolactone), poly hydroxy alka-noates, poly(hydroxyl butyrate) and poly(hydroxyl valerate).

4. The material according to claim 1, wherein the recombinant human collagen is selected from the group consisting of recombinant human collagen type I, II, III, V, VI, IX and XI.

5. The material according to claim 1 wherein the recombinant human collagen is freeze-dried.

6. The material according to claim 1 wherein the recombinant human collagen is cross-linked.

7. The material according to claim 1 wherein the mesh comprises polymer fibres of diameter of from 5 to 100 μm.

8. The material according to claim 1 wherein the mesh and/or the collagen material have a porosity of 85-99%.

9. The material according to claim 1 wherein the thickness of the material is from 0.1 to 50 mm.

10. (canceled)

11. A medical device comprising the material of claim 1.

12. The medical device according to claim 11 further comprising cells, preferably selected from cartilage forming cells.

13. (canceled)

14. A method of manufacturing a material according to claim 1 any one of claims 1-9 or a medical device according to claim 11 or 12, the method comprising:
(i) obtaining carded mesh comprising one or more synthetic polymers;
(ii) needle punching the carded mesh to form a needle punched carded mesh;
(iii) obtaining recombinant human collagen; and
(iv) admixing the needle punched carded mesh and the recombinant human collagen.

15. The method according to claim 14 further comprising freeze-drying the recombinant human collagen.

16. The method according to claim 15 further comprising cross-linking the freeze-dried recombinant human collagen.

17. A three dimensional material produced by the method according to claim 1.

18. A method of repairing cartilage lesions, or postponing or eliminating the expansion of a cartilage lesion in a subject in need thereof, said method comprising
(i) providing a medical device comprising a three dimensional porous material comprising needle punched carded mesh comprising one or more synthetic polymers, and recombinant human collagen, and
(ii) applying the medical device to the lesion site of a subject.

19. The method according to claim 18, wherein the subject is a human patient or an animal.

20. The method according to claim 18, wherein the lesion site is selected from elastic cartilage, hyaline cartilage and fibrocartilage.

21. The method according to claim 20, wherein the cartilage is in a synovial joint selected from the group consisting of a hip, knee, ankle, elbow, shoulder, finger, toe, wrist and temporomandibular joint.
